(19) United States
(12) Patent Application Publication
(10) Pub. No.: US 2011/0009497 A1
(43) Pub. Date: Jan. 13, 2011

(54) DRUG-CONTAINING COMPOSITION

(75) Inventors: Kentaro Nakamura, Ashigarakami-gun (JP); Shouji Ooya, Ashigarakami-gun (JP); Tetsuo Hiratou, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Minato-ku, Tokyo (JP)

(21) Appl. No.: 12/933,631

(22) PCT Filed: Mar. 18, 2009
(86) PCT No.: PCT/JP2009/055248

§ 371 Date: Sep. 20, 2010

(30) Foreign Application Priority Data
Mar. 21, 2008 (JP) 2008-073140

(51) Int. Cl. A61K 47/42

(52) U.S. Cl. 514/774; 514/773; 514/776

(57) ABSTRACT

It is an object of the present invention to provide a drug-containing composition which is capable of dissolving a poorly water-soluble drug, has low toxicity to the human body, and has high binding affinity with drugs. The present invention provides a composition which is composed of: (a) at least one poorly water-soluble compound; and (b) a carrier comprising a polymer (excluding plasma protein) having binding affinity with the poorly water-soluble compound.

### TECHNICAL FIELD

The present invention relates to a novel composition capable of solubilizing a water-insoluble or poorly water-soluble compound (preferably a pharmaceutically active ingredient) in water. When the composition of the present invention is applied to a pharmaceutical composition, the effective administration dose can be reduced, so that an effect can be achieved that adverse effects of the pharmaceutically active ingredient is reduced.

### BACKGROUND ART

Even if pharmaceutically active ingredients have strong bioactivity, they cannot exhibit their effects if they are poorly water-soluble. Therefore, in the pharmaceutical industry, there are many preparations the development of which has been abandoned or which have been marketed while exhibiting the activity at lower levels than original activity levels.

As a method for solubilizing a water-insoluble or poorly water-soluble pharmaceutically active ingredient, the following methods (A) to (C) exist.
(A) A method comprising partially altering the structure of a medicinal substance so as to obtain a soluble derivative: a soluble derivative such as hydrochloride, hydrobromate, sulfate, methanesulfonate, sodium salt, potassium salt, or sodium sulfonate is obtained.
(B) A method comprising adding a solubilizing agent: addition of a surfactant causes micellization and emulsification for solubilization, or serum albumin or plasma protein is used.
(C) A method comprising using an organic solvent alone or a mixed solvent of an aqueous solvent and an organic solvent: propylene glycol or the like is used for solubilization.

However, in the case of method (A) above, the structure of a pharmaceutical drug substance serving as an active ingredient is partially altered. Therefore, the solubility of a drug substance itself cannot be increased. In addition, when a drug is obtained in the form of a derivative, a variety of problems arise. Such problems include reduction of the activity of the obtained pharmaceutical product itself and precipitation of a medicinal substance due to changes in pH. Thus, such method is not a desirable method.

Among the method (B) above, in the method which involves the use of a surfactant, there are actually very few surfactants that are biologically safe and exhibit effective soluble properties. There is an example of such method wherein a medicinal substance (Taxol) is dissolved using polyoxyethylated castor oil (Cremophor EL). However, it has been reported that polyoxyethylated castor oil can cause rouleaux formation of red blood cells (Non-Patent Document 1). Since there are very few safe and useful surfactants, formulations obtained by dissolving paclitaxel or cyclosporine with the use of a toxic Cremophor have been used.

In addition, in the case of method (C) above, which involves the use of an organic solvent such as propylene glycol, there are very few safe organic solvents that are inert in terms of bioactivity and do not cause hemolysis. Therefore, the method is less likely to be used in practice in the field of medicine. For instance, Patent Documents 1 and 2 each describe a production method comprising a step of dissolving a poorly water-soluble dihydropyridine composition in an organic solvent or a mixed solvent of water and an organic solvent. However, the thus obtained solution is a turbid solution in which partial precipitation can be confirmed to take place. Therefore, it is understood that the composition is not sufficiently solubilized. This indicates precipitation of poorly water-soluble pharmaceutically active ingredients, showing that sufficient activity cannot be exhibited and that in vivo toxicity resulting from such precipitation is not improved.

Further, in recent years, as a method for solubilizing a poorly water-soluble drug, a method for solubilizing such drug with the use of serum albumin, which corresponds to method (B) above, has been used in some cases. However, in the case of a method for solubilizing a poorly water-soluble drug with the use of serum albumin, serum albumin binds to the drug via nonspecific adsorption. In such case, the binding affinity is poor (dissociation constant Kd=10⁻⁵ to 10⁻³M; it is known that the dissociation constant is approximately several tens of micro-moles (micro-molarity) (μM), even in the case of binding between albumin and warfarin that is thought to be strong). Therefore, the drug is likely to dissociate from albumin, which is a serious drawback. This makes it difficult to adequately transport the drug to biological molecules that are targets of the drug (hereinafter referred to as “disease molecules”). As a result, in order to deliver an effective dose of the drug to disease molecules, the option of increasing the administration dose of the drug is an unavoidable choice. Therefore, as naturally expected, adverse effects of the drug are intensified, resulting in increased burdens and risks for a patient, which is seriously problematic.

Patent Document 3 describes an example of method (B) above, wherein a pharmaceutical composition comprising a poorly water-soluble compound having substantial binding affinity with a plasma protein is used. However, a pharmaceutical composition used for such composition needs to have substantial affinity with a specific plasma protein to be used. Therefore, no universal solutions for the aforementioned problem can be obtained.
[Non-Patent Document 1] The Lancet, vol. 352: 540-542
[Patent Document 1] Hungarian Patent No. 198381
[Patent Document 2] German Patent No. 3702105
[Patent Document 3] Japanese Patent Publication (Kohyo) No. 2000-508806 A

### DISCLOSURE OF THE INVENTION

### Problem to be Solved by the Invention

In the cases of the conventional techniques, in order to dissolve a poorly water-soluble drug and to deliver the drug, a method comprising using a surfactant, an organic solvent, or a drug vehicle such as serum albumin for nonspecific adsorption has been used as described above. However, the use of a surfactant or an organic solvent is problematic in terms of toxicity to the human body inherent to a surfactant or an organic solvent. In addition, in the case of a method comprising using a drug vehicle such as serum albumin for nonspecific adsorption, since albumin binds to a drug via nonspecific adsorption, the binding affinity is poor. Accordingly, the drug is likely to dissociate from albumin, which is a serious drawback. This makes it difficult to adequately transport the drug to biological molecules that are the targets of the drug (hereinafter referred to as “disease molecules”). As a result, in order to deliver the effective dose of the drug to disease molecules, the option of increasing the administration dose of the drug is an unavoidable choice. Therefore, as naturally expected, adverse effects of the drug are intensified, resulting in increased burdens and risks for a patient, which is seriously problematic. Specifically, it is an object of the present invention to provide a drug-containing composition which is capable of dissolving a poorly water-soluble drug, has low toxicity to the human body, and has high binding affinity with drugs.

### Means for Solving Problem

As a result of intensive studies in order to attain the above object, the present inventors found that the risk of dissociation between a drug carrier and a drug, which is problematic when albumin is used as a drug carrier, can be significantly reduced with the use of a biopolymer having high binding affinity with the drug as a drug carrier for delivering a poorly water-soluble drug. This has led to the completion of the present invention.

Thus, the present invention provides a composition which is composed of: (a) at least one poorly water-soluble compound; and (b) a carrier comprising a polymer (excluding plasma protein) having binding affinity with the poorly water-soluble compound.

Preferably, the polymer having binding affinity with the poorly water-soluble compound is a polymer having binding affinity that is a dissociation constant Kd of 10⁻⁶ to 10⁻¹⁵ M, more preferably 10⁻⁸ to 10⁻¹⁴ M, particularly preferably 10⁻⁹ to 10⁻¹³ M with the poorly water-soluble compound.

Preferably, the poorly water-soluble compound is a pharmaceutical product.

Preferably, the polymer having binding affinity with the poorly water-soluble compound is a protein.

Preferably, the protein is: a protein containing an amino acid sequence of a receptor of a poorly water-soluble compound, a sequence responsible for binding which is contained in a receptor of a poorly water-soluble compound, an amino acid sequence of an antibody to a poorly water-soluble compound, or a sequence responsible for binding which is contained in an antibody to a poorly water-soluble compound; a protein that binds to a poorly water-soluble compound; or a protein containing a sequence responsible for binding which is contained in a protein that binds to a poorly water-soluble compound.

Preferably, the protein is a protein which was produced by gene recombinant techniques.

Preferably, a different protein is further bound directly or via a linker to the N-terminal and/or the C-terminal of the protein.

Preferably, the different protein binding to the N-terminal and/or the C-terminal of the protein is a protein that can control the release of a poorly water-soluble compound by causing a steric hindrance or a protein that functions in vivo as a scaffold.

Preferably, the protein that functions in vivo as a scaffold is gelatin, collagen, albumin, elastin, or fibrin.

Preferably, the composition of the present invention is a pharmaceutical composition for administering the poorly water-soluble compound to patients.

### Effects of the Invention

According to the present invention, the present inventors succeeded in significantly reducing the risk of dissociation between a drug carrier and a drug, which is problematic when albumin is used as a drug carrier, with the use of a biopolymer having high binding affinity with the drug as a drug carrier for delivering a poorly water-soluble drug. As a result, the drug administration dose at which effective activity is exhibited can be reduced, thereby significantly reducing adverse effects inherent to the drug in a successful manner.

### BEST MODE FOR CARRYING OUT THE INVENTION

Hereinafter, the present invention is described in detail.

The composition of the present invention is characterized in that it is composed of: (a) at least one poorly water-soluble compound; and (b) a carrier comprising a polymer (excluding plasma protein) having binding affinity with the poorly water-soluble compound. The term “binding affinity” used herein refers to, for example, a specific non-covalent binding interaction, such as an enzyme-substrate, ligand-receptor, or enzyme-coenzyme interaction, which is susceptible to competitive inhibition caused by an adequate competitive molecule. In the present invention, the dissociation constant Kd for the binding between a poorly water-soluble compound and a carrier is preferably 10⁻⁶ to 10⁻¹⁵ M, more preferably 10⁻⁸ to 10⁻¹⁴ M, and particularly preferably 10⁻⁹ to 10⁻¹³ M.

FIG. 1 shows a typical example of the structure of the composition of the present invention. Drug a (poorly water-soluble compound), Protein A (a polymer having binding affinity with a poorly water-soluble compound), Protein B, Protein C, Linker A, and Linker B shown in FIG. 1 are described below.

<Drug “a”>

In the present invention, for example, a poorly water-soluble compound disclosed in PCT/JP/2007/066779 can be used as Drug “a”, which is a poorly water-soluble compound. Any poorly water-soluble compound may be used as long as it is a poorly water-soluble compound such as a pigment or a drug. In general, as an indicator of the hydrophilicity/hydrophobicity of a compound, the log of the 1-octanol/water partition coefficient (Log P) obtained by a flask shaking method (buffer solution: pH 7.4) has been widely used. It is also possible to obtain such value by calculation instead of actual measurement. (Log P used herein is calculated by the C LOG P program for the Hansch-Leo fragmental method, which is included in the “PCModels” system (Daylight Chemical Information Systems.)

The Log P of a poorly water-soluble compound used in the present invention is preferably 1 to 20, more preferably 1 to 15, particularly preferably 2 to 10, and most preferably 3 to 5.

A drug used herein comprises a physiologically active ingredient. Specific examples of a drug that can be used include marketed drugs such as Lipitor that is a therapeutic agent for hyperlipidemia and clopidogrel that is a platelet aggregation inhibitor, immunosuppressive agents (e.g., rapamycin, tacrolimus, and cyclosporine), anticancer agents (e.g., paclitaxel, Topotecin, taxotere, docetaxel, enocitabine, and 17-AAG), antipyretic analgesics (e.g., aspirin, acetaminophen, and sulpyrine), antiepileptic agents (e.g., phenytoin, acetazolamide, carbamazepine, clonazepam, diazepam, and nitrazepam), antiphlogistic analgetics (e.g., alclofenac, alminoprofen, ibuprofen, indomethacin, epirizole, oxaprozin, ketoprofen, diclofenac sodium, diflunisal, naproxen, piroxicam, fenbufen, flufenamic acid, flurbiprofen, floctafenine, pentazocine, metiazinic acid, mefenamic acid, and mofezolac), fat-soluble vitamins (e.g., vitamin A, vitamin D2, vitamin D3, vitamin E, and vitamin K2), synthetic antibacterial agents (enoxacin, ofloxacin, cinoxacin, sparfloxacin, thiamphenicol, nalidixic acid, tosufloxacin tosilate, norfloxacin, pipemidic acid trihydrate, piromidic acid, fleroxacin, and levofloxacin), antifungal agents (e.g., itraconazole, ketoconazole, fluconazole, flucytosine, miconazole, and pimaricin), antibiotics (e.g., roxithromycin, cefditoren pivoxil, cefteram pivoxil, erythromycin, clarithromycin, telithromycin, and azithromycin), antivirals (acyclovir, ganciclovir, didanosine, zidovudine, and vidarabine), hormone drugs (e.g., insulin zinc, testosterone propionate, and estradiol benzoate), cardiovascular agents (e.g., alprostadil), antithrombotic agents, gastrointestinal agents (omeprazole, lansoprazole, teprenone, metoclopramide, and sofalcone), diabetic agents (e.g., pioglitazone hydrochloride), antioxidants, antiallergic agents (clemastine fumarate, loratadine, mequitazine, zafirlukast, pranlukast, ebastine, tazanolast, tranilast, ramatroban, and oxatomide), steroidal anti-inflammatory agents (e.g., cortisone acetate, betamethasone, prednisolone, fluticasone propionate, dexamethasone, budesonide, beclometasone propionate, triamcinolone, loteprednol, fluorometholone, difluprednate, mometasone furoate, clobetasol propionate, diflorasone diacetate, diflucortolone valerate, fluocinonide, amcinonide, halcinonide, fluocinolone acetonide, triamcinolone acetonide, flumetasone pivalate, and clobetasone butyrate), cosmetic components, sulfa drugs (e.g., salazosulfapyridine, sulfadimethoxine, sulfamethizole, sulfamethoxazole, sulfamethopyrazine, and sulfamonomethoxine), anesthetic agents (e.g., fentanyl), ulcerative colitis therapeutic agents (e.g., mesalazine), and supplement components.

### <Protein A>

Protein A (a polymer having binding affinity with a poorly water-soluble compound) is a protein having affinity with Drug a. A receptor, a target protein, or a binding protein of Drug a can be used. Examples thereof include a vitamin D3 receptor, HMG-CoA reductase, an ADP receptor (P2Y12), a type-L calcium channel, a proton pump, a serotonin receptor, a dopamine receptor, a dopamine D2 receptor, an angiotensin II receptor, a melatonin MT1/MT2 receptor, an α2δ subunit of voltage-dependent calcium channel, PDGFR-α, PDGFR-β, VEGFR1, VEGFR2, VEGFR3, KIT, FLT3, CSF-1R, RET, a ribosome 50S subunit, Tubulin, DNA helicase, RNA polymerase, an acetylcholine receptor, a G protein conjugated receptor, a muscarinic acetylcholine receptor, an adenosine receptor, an adrenaline receptor, a GABA receptor (type B), an angiotensin receptor, a cannabinoid receptor, a cholecystokinin receptor, a glucagon receptor, a histamine receptor, an olfactory receptor, an opioid receptor, rhodopsin, an secretin receptor, a somatostatin receptor, a gastrin receptor, an erythropoietin receptor, an insulin receptor, a cell growth factor receptor, a cytokine receptor, a guanylate cyclase receptor, a GC-A, GC-B, or GC-C guanylin receptor, a nicotinic acetylcholine receptor, a glycine receptor, a GABA receptor (type A or C), a glutamic acid receptor, a type-3 serotonin receptor, inositol triphosphate (IP3) receptor, ryanodine receptor, a steroid hormone receptor, a sex hormone (androgen, estrogen, or progesterone) receptor, a vitamin D receptor, a glucocorticoid receptor, a mineralocorticoid receptor, a thyroid hormone receptor, a retinoid receptor, a peroxisome proliferator-activated receptor (PPAR), an insect molting hormone (ecdysone) receptor, a dioxin receptor (AhR), and a benzodiazepine receptor.

Protein A may be a naturally occurring biologically derived protein or a protein produced by gene recombination technology. However, with regard to the designing described below, a protein produced by gene engineering is preferable. Such protein may comprise a naturally occurring sequence or a sequence newly designed depending on application. As a sequence newly designed depending on application, a sequence substantially responsible for binding extracted from a naturally derived sequence of the protein, which is directly or indirectly essential for the binding to Drug “a” can be used. In addition, as a newly designed sequence, a sequence obtained by partially altering the amino acid sequence contained in a natural sequence of the protein can be used. Specifically, an amino acid sequence of the protein or an amino acid sequence contained in a sequence responsible for binding extracted from the protein can be adjusted, thereby adjusting the solubility of the protein or interaction between the protein and a different biologically derived molecule. In addition, a side chain that is contained in a sequence responsible for the binding to Drug “a” and is directly or indirectly involved in the binding to Drug “a” can be substituted with a different side chain, thereby attenuating or intensifying the affinity. Such substitution can be carried out in a manner such that the protein sequence is partially altered or 1 to 50 residue(s) are newly inserted into or deleted from the protein sequence.

Further, the above protein may be chemically modified in vivo or in vitro. For instance, chemical modification of amino groups in the protein that can be carried out includes, but is not limited to, formation of guanidyl, amidin, or reduced alkyl, carbamylation, acetylation, succinylation, maleylation, acetoacetylation, formation of nitrotroponyl, deaminization, modification with a carbonyl compound, dinitrophenylation, and/or trinitrophenylation. In addition, chemical modification of carboxyl groups contained in the protein that can be carried out includes, but is not limited to, amidation and/or esterification. Further, for chemical modification, modification with sugar chains may be carried out.

Furthermore, the above protein may contain an auxiliary molecule that allows the three-dimensional structure to be maintained, the ability to bind to a ligand or substrate to be secured, or the in vivo stability or physiological functions to be maintained. Examples of such auxiliary molecules that can be used include Zn, Fe, Cd, Cu, Au, Ag, Pt, Hg, Na, Cl, K, Ca, Li, Mg, Al, Co, Mn, Cr, Ga, Ge, Ni, Br, Rb, Mo, and Pb atoms or molecules, complexes (e.g., heme and protoheme complexes) comprising such atoms or molecules, and ions or complex ions thereof. In addition, a coenzyme, an electron carrier, or the like can be used as such auxiliary molecule. Specific examples thereof include, but are not limited to, quinone, pyrroloquinoline quinone, top a quinone, tryptophan-tryptophylquinone, lycine tyrosyl quinone, cystenyl-tryptophanquinone, thiamine diphosphate, coenzyme A (pantothenic acid), coenzyme R (biotin), coenzyme F (folic acid), ATP (adenosine triphosphate), uridine diphosphate glucose, NAD⁺/NADH (nicotinamide adenine dinucleotide), FMN/FMNH₂ (flavin mononucleotide), FAD/FADH₂ (flavin adenine dinucleotide), ubiquinone, cytochrome, NADP⁺/NADPH (nicotinamide adenine dinucleotide phosphate), plastoquinone, plastocyanin, ferredoxin, chlorophyll, pheophytin, thioredoxin, menaquinone, caldariellaquinone, coenzyme F₄₂₀, rhodoquinone, Riske, and Blue-Cu.

### <Protein B>

A different protein (namely, Protein B) can be bound to Protein A.

A variety of structural proteins or structural peptides can be used as Protein B that can be bound to Protein A. For instance, Protein B can regulate the release of Drug “a” by causing a steric hindrance. Specifically, in order to control the rate of release of Drug “a” from the sequence domain responsible for binding or the proportion of released Drug “a”, a different structural protein sequence that can serve as a “cap” in the steric structure (hereinafter referred to as “cap protein sequence”) can be used as Protein B. That is, it is possible to design a sequence that can serve as a cap in the three-dimensional structure and to use such sequence with Protein A. In addition, examples of such cap protein sequence that can be used include GIGDPVTCLKSGAICHPVFCPRRYKQIGTCGLPGTKCCKK (each letter denoting a single amino acid). Also, a protein sequence having unique functions can be used as Protein B. Such Protein B having unique functions can be modified depending on application and is not particularly limited. For example, a sequence having a function to exhibit antibacterial activity, blood sugar regulatory activity, activity of regulating the urge to eat, blood pressure regulatory activity, analgesic activity, antiviral activity, anticoagulating activity, vasoconstriction/vasodilatation activity, tranquilizing activity, antidepressive activity, mental exaltation activity, or adhesion activity can be used. More specific examples of such sequence include antibacterial peptides, defensin, lactoferricin, magainin, tachyplesin, angiotensin, bradykinin, T kinin, fibrinopeptides, natriuretic peptides (for atrial or cerebral natriuresis), urodilatin, guanine, uroguanine, endothelin, big endothelin, salusin, urotensin, oxytocin, vasopressin, neurophysin, proopiomelanocortin-derived peptides, posterior pituitary hormone, adrenocorticotropic hormone, corticotropin-like intermediate-lobe peptide, endorphin, lipotropin, melanocyte-stimulating hormone, hypothalamic hormone, urocortin, somatostatin, cortistatin, TRH, prolactin, pituitary adenylate cyclase-activating peptide, metastin, tachykinin, substance P, neuropeptide, neurokinin, endokinin, neurotensin, neuromedin, ghrelin, obestatin, agouti-related protein, melanin-concentrating hormone, neuropeptide, orexin, opioidpeptide, dynorphin, neoendorphin, leumorphin, methionine enkephalin, leucine enkephalin, methionine enkephalin, adrenorphin, endomorphin, nociceptin, orphanin, nocistatin, RFamide peptide, galanin, gastrin, cholecystokinin, motilin, pancreatic polypeptide, gastric inhibitory peptide, peptide YY, peptide HM, vasoactive intestinal peptide, secretin, apelin, insulin, C peptide, insulin-like peptide, relaxin, relaxin-like peptide, glucagon, glicentin, glucagon-like peptide, oxyntomodulin, CGRP, adrenomedullin, proadrenomedullin, calcitonin receptor-stimulating peptide, amyrin, calcitonin, catacalcin, parathyroid hormone, cathelicidin, thymosin, and humanin.

In addition, in order to allow Protein B to pass through the blood-brain barrier, a peptide such as microglia-derived brain transfer polypeptide sequence described in WO2005/014625 (International Application No.: PCT/JP2004/011668) that can pass through the blood-brain barrier can be used as Protein B. Protein A and Protein B may be directly bound to each other, or they may be bound to each other via a linker (hereinafter referred to as Linker A).

Linker A is not particularly limited, as long as it binds Protein A and Protein B. Preferably, a versatile linker sequence or a linker designed for specific purposes can be used in the form of a protein sequence containing peptide bonds. As a versatile linker, a peptide comprising 2 to 40 residues can be used. In order to obtain a linker designed for a specific purpose, a linker can be designed in accordance with such purpose and is not particularly limited. However, a sequence that is cleaved in vivo in the presence of protease activity, a sequence that is phosphorylated by a certain factor, a sequence that is hydrolyzed, a sequence containing a sequence to be methylated, or the like can be used. More specifically, a sequence that is cleaved by a blood-clotting factor protease or a sequence that is cleaved by a matrix metalloprotease can be used. However, the above linker is not limited to such examples. As examples of a sequence that is cleaved by thrombin, the sequences described in the following can be used: Thrombin specificity, Requirement for apolar amino acids adjacent to the thrombin cleavage site of polypeptide substrate, Jui-Yoa CHANG. Eur. J. Biochem. 151, 217-224 (1985) FEBS (Factor Xa, prothrombin, or FactorVII); and X-ray Structure of Active Site-inhibited Clotting Factor Xa, IMPLICATIONS FOR DRUG DESIGN AND SUBSTRATE RECOGNITION, Hans Brandstetter, et. al. Volume 271, Number 47, Issue of Nov. 22, 1996 pp. 29988-29992, THE JOURNAL OF BIOLOGICAL CHEMISTRY. For example, the sequence LVPRGSIEGR (each letter denoting a single amino acid) can be used.

### <Protein C>

A different protein, namely Protein C, can be bound to Protein A or Protein B described above.

A variety of structural proteins and structural peptides can be used as Protein C. For instance, a protein sequence that functions in vivo as a scaffold can be designed and used. Protein C is not limited as long as it is a protein that can function as a scaffold. Examples of Protein C that can be used include gelatin, collagen, albumin, elastin, and fibrin. In addition, Protein C may be a natural biologically derived substance or a gene recombinant.

Protein C may be bound directly or via a linker (hereinafter referred to as Linker B) to Protein A or Protein B.

Linker B is not particularly limited, as long as it binds Protein A (or Protein B) and Protein C. Preferably, a versatile linker sequence or a linker designed for specific purposes can be used in the form of a protein sequence containing peptide bonds. As a versatile linker, a peptide comprising 2 to 40 residues can be used. In order to obtain a linker designed for a specific purpose, a linker can be designed in accordance with such purpose and is not particularly limited. However, a sequence that is cleaved in vivo in the presence of protease activity, a sequence that is phosphorylated, a sequence that is hydrolyzed, a sequence containing a sequence to be methylated, or the like can be used. More specifically, a sequence that is cleaved by a blood-clotting factor protease or a sequence that is cleaved by a matrix metalloprotease can be used. However, the above linker is not limited to such examples. As examples of a sequence that is cleaved by thrombin, the sequences described in the following can be used: Thrombin specificity, Requirement for apolar amino acids adjacent to the thrombin cleavage site of polypeptide substrate, Jui-Yoa CHANG. Eur. J. Biochem. 151, 217-224 (1985) FEBS (Factor Xa, prothrombin, or FactorVll); and X-ray Structure of Active Site-inhibited Clotting Factor Xa. IMPLICATIONS FOR DRUG DESIGN AND SUBSTRATE RECOGNITION, Hans Brandstetter, et. al. Volume 271, Number 47, Issue of Nov. 22, 1996 pp. 29988-29992, THE JOURNAL OF BIOLOGICAL CHEMISTRY. For example, the sequence LVPRGSIEGR (each letter denoting a single amino acid) can be used.

Known methods can be used to cause the expression of the proteins described above and to produce such proteins.

The use of the composition of the present invention is not particularly limited. However, the composition can be used for therapeutic drugs for a variety of diseases, and therefore it can be used as a topical therapeutic agent, an oral therapeutic agent, a parenteral injection, or the like.

The present invention is hereafter described in greater detail with reference to the following examples, although the present invention is not limited thereto.

### EXAMPLES

### Example 1

The experiments described below were carried out using vitamin D3, which is a poorly soluble compound known to promote bone regeneration.

A human vitamin D3 receptor was expressed with the use of Escherichia coli BL21 (DE3) Codon-plus as a His-tag fusion protein (using a vector (pQE30 Xa; QIAGEN)). For culture, an LB (Luria-Bertani) medium containing 100 μg/ml ampicillin was used. Preculture was carried out with the use of a 300-mL LB medium contained in a 500-mL Erlenmeyer flask at 37° C. Then, for main culture, 30 mL of the preculture solution was added to a 1.5-L LB medium (containing 100 μg/ml ampicillin) contained in a 3-L baffled Erlenmeyer flask and subjected to shake culture at 37° C. so as to result in 0.6 OD600. Thereafter, IPTG was added thereto to a final concentration of 0.5 mM for expression induction, followed by overnight shake culture at 30° C. Subsequently, cells were collected by centrifugation and washed. The obtained bacterial cells were suspended in 200 mM NaCl, 50 mM sodium phosphate buffer, and 10 mM imidazole (pH 8.0), followed by ultrasonic disintegration for 5 minutes and centrifugation at 44,200×g for 30 minutes. Thus, the supernatant was obtained. The obtained supernatant was introduced at a flow rate of 0.1 ml/min into an Ni-column (Ni-NTA His-Bind Resin: Novagen; column volume: 50 ml) that had been preliminarily equilibrated with a solution A (300 mM NaCl, 50 mM sodium phosphate buffer, 20 mM imidazole, pH 8.0) for immobilization. The column was washed with 500 ml of a solution B (300 mM NaCl, 50 mM sodium phosphate buffer, 20 mM imidazole, pH 8.0), followed by elution with a solution C (300 mM NaCl, 50 mM sodium phosphate buffer, 250 mM imidazole, pH 8.0). Further, the eluate was subjected to gel filtration chromatography (with the use of a Superdex 75 10/300 GL column (GE); buffer: solution A) with the use of AKTA FPLC. High-purity fractions were exclusively collected, followed by dialysis/concentration. Eventually, a His-tag fusion vitamin D3 receptor protein dissolved in the final solution A was obtained.

Vitamin D3 (5 mg) was bound to 1000 ml of a His-tag fusion vitamin D3 receptor protein (0.5 mg/ml). A portion (10 ml) of the resultant was introduced at a flow rate of 0.05 ml/min into an Ni-column (Ni-NTA His-Bind Resin (Novagen); column volume: 10 ml) that had been preliminarily equilibrated with a solution A (300 mM NaCl, 50 mM sodium phosphate buffer, 20 mM imidazole, pH 8.0) for immobilization. Further, the Ni-column was washed with 50 ml of a solution A. Human serum (20 ml) was introduced into the column at a flow rate of 0.1 ml/min. Then, a solution A (10 ml) was introduced thereinto at a flow rate of 0.1 ml/min.

Vitamin D3 that had been eluted from the column was quantified by high-performance liquid chromatography HPLC (the column used: Wakosil 5-SIL). As a result, the amount of the eluate was confirmed to merely correspond to approximately 5% of the total amount of bound vitamin.

Herein, the dissociation constant Kd for a vitamin D3 receptor protein and vitamin D3 was 2.2×10⁻⁹±5.6×10⁻⁹ M.

### Comparative Example 1

A human albumin sequence was expressed with the use of Escherichia coli BL21 (DE3) Codon-plus as a His-tag fusion protein (using a vector (pQE30 Xa; QIAGEN)). For culture, an LB (Luria-Bertani) medium containing 100 μg/ml ampicillin was used. Preculture was carried out with the use of a 300-mL LB medium contained in a 500-mL Erlenmeyer flask at 37° C. Then, for main culture, 30 mL of the preculture solution was added to a 1.5-L LB medium (containing 100 μg/ml ampicillin) contained in a 3-L baffled Erlenmeyer flask and subjected to shake culture at 37° C. so as to result in 0.6 OD600. Thereafter, IPTG was added thereto to a final concentration of 0.5 mM for expression induction, followed by overnight shake culture at 30° C. Subsequently, cells were collected by centrifugation and washed. The obtained bacterial cells were suspended in 200 mM NaCl, 50 mM sodium phosphate buffer, and 10 mM imidazole (pH 8.0), followed by ultrasonic disintegration for 5 minutes and centrifugation at 44,200×g for 30 minutes. Thus, the supernatant was obtained. The obtained supernatant was introduced at a flow rate of 0.1 ml/min into an Ni-column (Ni-NTA His-Bind Resin: Novagen; column volume: 50 ml) that had been preliminarily equilibrated with a solution A (300 mM NaCl, 50 mM sodium phosphate buffer, 20 mM imidazole, pH 8.0) for immobilization. The column was washed with 500 ml of a solution B (300 mM NaCl, 50 mM sodium phosphate buffer, 20 mM imidazole, pH 8.0), followed by elution with a solution C (300 mM NaCl, 50 mM sodium phosphate buffer, 250 mM imidazole, pH 8.0). Further, the eluate was subjected to gel filtration chromatography (with the use of a Superdex 200 10/300 GL column (GE); buffer: solution A) with the use of AKTA FPLC. High-purity fractions were exclusively collected, followed by dialysis/concentration. Eventually, a His-tag fusion albumin receptor protein dissolved in the final solution A was obtained.

Vitamin D3 (5 mg) was bound to 1000 ml of a His-tag fusion albumin receptor protein (0.5 mg/ml). A portion (10 ml) of the resultant was introduced at a flow rate of 0.05 ml/min into a Ni-column (Ni-NTA His-Bind Resin (Novagen); column volume: 10 ml) that had been preliminarily equilibrated with a solution A (300 mM NaCl, 50 mM sodium phosphate buffer, 20 mM imidazole, pH 8.0) for immobilization. Further, the Ni-column was washed with 50 ml of a solution A. Human serum (20 ml) was introduced into the column at a flow rate of 0.1 ml/min. Then, a solution A (10 ml) was introduced thereinto at a flow rate of 0.1 ml/min. Human serum (20 ml) was introduced into the Ni-column at a flow rate of 0.1 ml/min. Thereafter, 10 ml of phosphate buffer (pH 7.0) was introduced thereinto at a flow rate of 0.1 ml/min.

Vitamin D3 that had been eluted from the column was quantified by high-performance liquid chromatography HPLC (the column used: Wakosil 5-SIL). As a result, the amount of the eluate was confirmed to correspond to approximately 70% of the total amount of bound vitamin.

As a result of comparison of Example 1 and Comparative Example 1 described above, it was confirmed that the composition of the present invention is less likely to dissociate in human serum than conventionally used albumin.

### Example 2

The His-tag fusion vitamin D3 receptor protein produced in Example 1 was again bound to an Ni-column as in the case of Example 1. Then, 1% (w/w) Factor Xa (GE Healthcare Bioscience) was added to the His-tag fusion vitamin D3 receptor protein. The resultant was allowed to stand still overnight at 22° C. and eluted with a solution A as in the case of Example 1. The eluate was introduced into a benzamidine column (GE Healthcare Bioscience; HiTrap Benzamidine FF (high sub) column) in a general purification step, during which a high-purity vitamin D3 receptor protein was purified. Accordingly, a drug carrier was obtained.

An excessive amount of 1α,25-dihydroxyvitamin D3 (calcitriol) (which is active vitamin D3) was dissolved with the produced drug carrier protein. The obtained liquid was administered 3 times per week via intravenous injection to osteoporosis model rats (ovariectomized aged rats) such that the calcitriol concentration was maintained at 0.3 μg/kg. Four months later, induction of bone regeneration in the rats was confirmed.

### BRIEF DESCRIPTION OF THE DRAWINGS

FIG. 1 shows a representative example of the structure of the composition of the present invention.

FIG. 2 shows an image of binding between vitamin D3 and the carrier of the present invention.

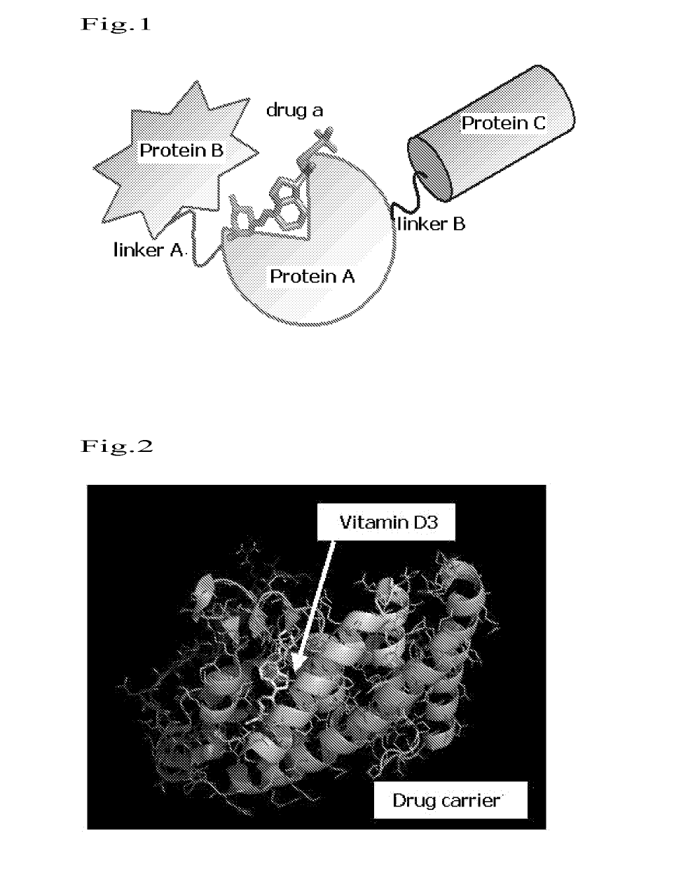

1. A composition which is composed of: (a) at least one poorly water-soluble compound; and (b) a carrier comprising a polymer (excluding plasma protein) having binding affinity with the poorly water-soluble compound.

2. The polymer according to claim 1, wherein the polymer having binding affinity with the poorly water-soluble compound is a polymer having binding affinity that is a dissociation constant Kd of 10⁻⁶ to 10⁻¹⁵ M with the poorly water-soluble compound.

3. The composition according to claim 1, wherein the poorly water-soluble compound is a pharmaceutical product.

4. The composition according to claim 1, wherein the polymer having binding affinity with the poorly water-soluble compound is a protein.

5. The composition according to claim 4, wherein the protein is: a protein containing an amino acid sequence of a receptor of a poorly water-soluble compound, a sequence responsible for binding which is contained in a receptor of a poorly water-soluble compound, an amino acid sequence of an antibody to a poorly water-soluble compound, or a sequence responsible for binding which is contained in an antibody to a poorly water-soluble compound; a protein that binds to a poorly water-soluble compound; or a protein containing a sequence responsible for binding which is contained in a protein that binds to a poorly water-soluble compound.

6. The composition according to claim 4, wherein the protein is a protein which was produced by gene recombinant techniques.

7. The composition according to claim 4, wherein a different protein is further bound directly or via a linker to the N-terminal and/or the C-terminal of the protein.

8. The composition according to claim 7, wherein the different protein binding to the N-terminal and/or the C-terminal of the protein is a protein that can control the release of a poorly water-soluble compound by causing a steric hindrance or a protein that functions in vivo as a scaffold.

9. The composition according to claim 8, wherein the protein that functions in vivo as a scaffold is gelatin, collagen, albumin, elastin, or fibrin.

10. The composition according to claim 1, which is a pharmaceutical composition for administering the poorly water-soluble compound to patients.


SEQUENCE LISTING (2 sequences)

SEQ ID NO: 1 - protein, 40 aa, Artificial Sequence
  Description of Artificial Sequence Synthetic peptide
GIGDPVTCLKSGAICHPVFCPRRYKQIGTCGLPGTKCCKK

SEQ ID NO: 2 - protein, 10 aa, Artificial Sequence
  Description of Artificial Sequence Synthetic peptide
LVPRGSIEGR